(19) United States
(12) Patent Application Publication
(10) Pub. No.: US 2001/0040990 A1
(43) Pub. Date: Nov. 15, 2001

(54) DISPLAY METHODS AND APPARATUS PARTICULARLY USEFUL FOR DISPLAYING SURGICAL WORKING OPERATIONS

(76) Inventor: MICHEL DADI, TEL AVIV (IL)

Correspondence Address: KENYON & KENYON, ONE BROADWAY, NEW YORK, NY, 10004, US

(21) Appl. No.: 09/060,545

(22) Filed: Apr. 15, 1998

(30) Foreign Application Priority Data
Apr. 15, 1997 (IL) 120667
Jun. 15, 1997 (IL) 121083

(51) Int. Cl. G06K 9/00

(52) U.S. Cl. 382/128

(57) ABSTRACT

A method of displaying a point of interest on an object, comprising preparing a reference pattern of the object, which reference pattern includes the point of interest in a visually-discernible manner on the object and further includes three visually-discernible reference points on the object, superposing the reference pattern on the object, effecting relative movement between the reference pattern and the object to optically align the three reference points on the reference pattern with three corresponding reference points on the subject and displaying the reference pattern superposed on the object such that the point of interest on the reference pattern is visually indicated on the object.

### FIELD AND BACKGROUND OF THE INVENTION

The present invention relates to display methods and apparatus particularly useful for displaying working operations to be performed on objects.

The invention in especially useful for displaying the location, direction, and/or magnitude a surgical operation is to be performed on a patient, such as drilling a hole in a patient's bone for purposes of implant surgery, and in therefore described below with respect to such an application. It will be appreciated, however, that the invention could also be used in many other surgical operations, as well as in many industrial or manufacturing operations, as described below.

The success of a surgical implant into a subject's bone depends to a high degree on the precision exercised in the location, direction, and/or depth of drilling an initial hole through the subject's bone for the implant, and thereby on the skill and experience of the person performing the drilling. This is particularly true in dental implants but also applies with respect to many other surgical procedures, as well as many industrial and manufacturing procedures requiring various operations to be precisely performed.

### OBJECTS AND BRIEF SUMMARY OF THE INVENTION

An object of the present invention is to provide novel display methods and apparatus having advantages, as will be described more particularly below.

A particular object of the present invention is to provide novel methods and display apparatus especially useful for assisting in the performance of various surgical, as well as non-surgical, procedures by providing an optical guide which displays the location, direction, and/or magnitude that a critical operation is to be performed. While the invention is particularly useful with respect to making dental implants, it can also be used in many other procedures, such as industrial and/or manufacturing processes requiring a critical working operation to be performed at a precise location, in a precise direction, and/or to a precise magnitude (e.g., depth of drilling a hole).

According to a broad aspect of the present invention, there is provided a method of displaying a point of interest on an object, comprising: preparing a reference pattern of the object, which reference pattern includes the point of interest in a visually-discernible manner on the object and further includes three visually-discernible reference points on the object; superposing the reference pattern on the object; effecting relative movement between the reference pattern and the object to optically align the three reference points on the reference pattern with three corresponding reference points on the object; and displaying the reference pattern superposed on the object such that the point of interest on the reference pattern is visually indicated on the object.

As will be described more particularly below, the invention is particularly useful wherein the object is a physical object, and the point of interest indicates the location, direction and/or magnitude a working operation is to be performed on the physical object.

According to one described preferred embodiment, the reference pattern is prepared in the form of a physical member having the configuration of the physical object. In this embodiment, the reference pattern is manipulated by manipulating the physical member.

A second embodiment is described wherein the reference pattern is prepared in the form of a computer-generated image having the configuration of the physical object. In this embodiment, the reference pattern is manipulated by electronically manipulating the computer-generated image.

A further embodiment is described below wherein the object is an image of the physical object, and the point of interest indicates the location of an element in the physical object.

The method of the present invention may be applied to display the object, and reference pattern superposed thereon, at a single station where a specific operation is to be performed. An additional advantage of the present invention, however, is that the object and the reference pattern superposed thereon may be displayed simultaneously at a plurality of stations, e.g., for instructional purposes.

As will be described below, the method of the present invention is particularly useful for performing surgical implant operations such as drilling a hole in a patient's jaw for purposes of implanting a dental device, or for performing other surgical operations, such as incision operations. However, the invention may also be used in non-surgical operations where a high degree of precision is required with respect to identifying the location of an element in the physical object, or in indicating the location, direction, and/or magnitude of a working operation, such as manufacturing or repairing precision parts, sculpturing, etc.

The invention also provides apparatus for displaying a point of interest in an object, or the location, direction, and/or magnitude a working operation is to be performed, in accordance with the above-described method.

Further features and advantages of the invention will be apparent from the description below.

### BRIEF DESCRIPTION OF THE DRAWINGS

The invention is herein described, by way of example only, with reference to the accompanying drawings, wherein:

FIG. 1 diagrammatically illustrates a simplified apparatus constructed in accordance with the present invention to aid in the performance of a working operation requiring a high degree of precision, in this case, the drilling of one or more holes through a patient's jaw for purposes of implanting a dental device;

FIG. 2 diagrammatically illustrates the apparatus of FIG. 1 wherein the reference pattern is computer-generated.

FIG. 3 diagrammatically illustrates a more sophisticated apparatus constructed in accordance with the invention also using a computer-generated image for the reference pattern rather than a physical member, and displaying the reference pattern superposed on the object at one or more stations;

FIG. 4 diagrammatically illustrates another apparatus constructed in accordance with the present invention to enable the viewer to selectively view the object from one side or the opposite side;

FIG. 4a illustrates the apparatus of FIG. 4 when viewed from the opposite side;

FIGS. 4b and 4c illustrate other arrangements that may be used with the apparatus of FIG. 4;

FIG. 5 diagrammatically illustrates further features in a preferred embodiment of the invention for fixing the object with respect to the reference pattern and the displaying device;

and FIG. 6 diagrammatically illustrates another apparatus constructed in accordance with the present invention in which the reference pattern is projected on an image of the object, rather than on the actual physical object.

### DESCRIPTION OF PREFERRED EMBODIMENTS

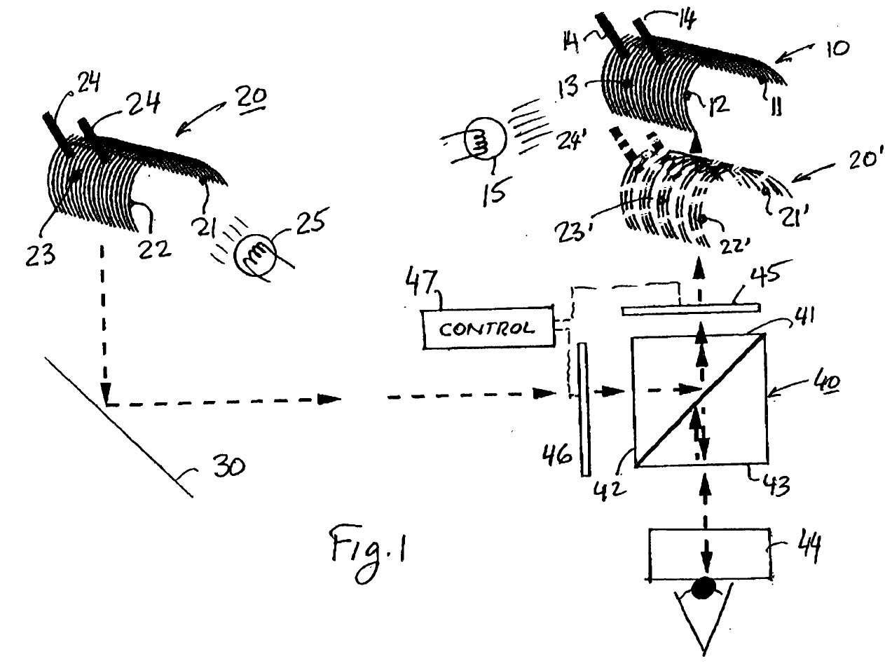

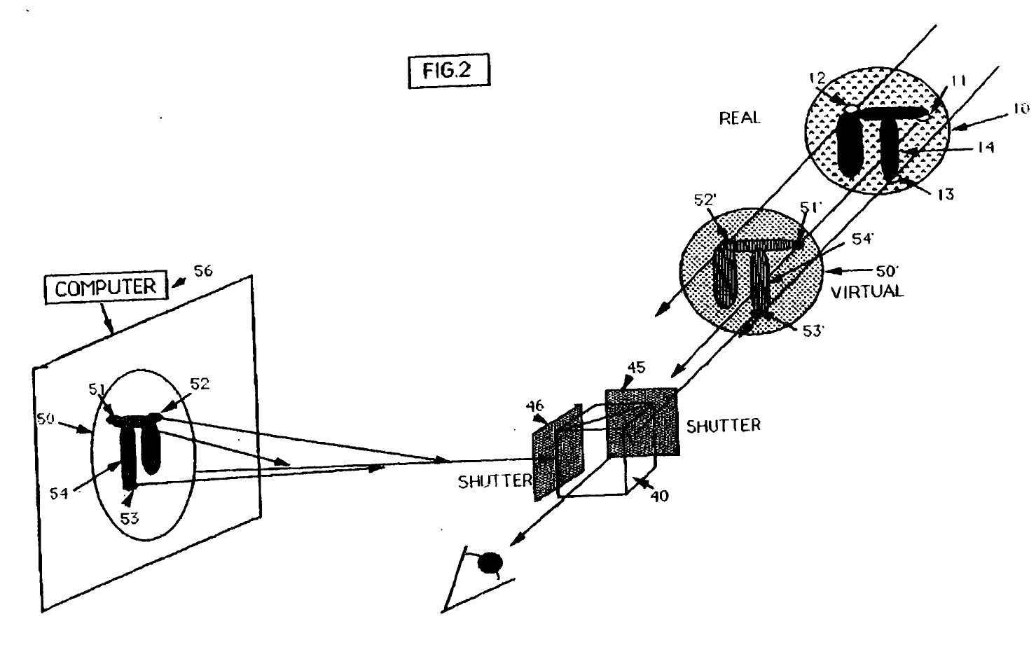

With reference first to FIG. 1, there is diagrammatically shown a simplified version of an apparatus constructed in accordance with the present invention for performing a working operation on an object, generally designated 10, requiring precision in the location, direction, and magnitude a working operation is to be performed on the object, In this case, the object 10 is the mouth of a subject, and the working operation to be performed is the drilling of one or more holes in the jaw of the subject each at a precise location, direction, and depth, for implanting a dental device.

For performing this working operation, a reference pattern, generally designated 20, is first constructed having a configuration corresponding to that of the object, i.e., the subject's jaw 10. Reference pattern 20 includes three visually-discernible reference points 21, 22, 23, and also one or more visually-discernible guides 24 indicating the location, direction, and depth each working (drilling of a hole) operation is to be performed on the object 10. For example, the reference points 21-23 may be simple opaque dots, and the guides 24 may be pins inserted into holes drilled at locations, orientations, and depths according to each hole to be drilled in the subject's jaw for implanting the dental device. If the pins are of known length, the viewable end of the pin protruding from the hole will indicate the depth of the hole to be drilled.

The reference pattern 20 is illuminated by a light source, schematically shown at 25, and its image is reflected by a 45° mirror 30 towards a beam splitter 40. Beam splitter 40 is used for projecting the virtual image 20′ of the reference pattern 20 onto the subject's jaw 10, and also for viewing the subject's jaw with the reference pattern superposed thereon. Thus, beam splitter 40 includes a first face 41 facing the subject's jaw 10, a second face 42 facing the 45° mirror 30 projecting the reference pattern, and a third face 43 for viewing the subject's jaw 10 with the image of the reference pattern 20′ superposed thereon. The illustrated apparatus further includes an eyepiece 44 aligned with the center of face 43 of the beam splitter 40 for viewing the subject's jaw 10 with the superposed reference pattern image 20′.

The three reference points 21-23 in the reference pattern 20 are selected so that corresponding points 11, 12 and 13 in the patient's jaw 10 can be easily identified and discerned when viewing the patient's jaw through the beam splitter 40. The subject's jaw is illuminated by a light source schematically indicated at 15.

After the reference pattern 20 has been optically projected onto the patient's jaw 10, the reference pattern is then manipulated to optically align the three reference points 21′-23′ of the projected image 20′ of the reference pattern with the three corresponding reference points 11-13 of the subject's jaw. Some adjustment may also be made of the mirror 30 and beam splitter 40, if desired. Since three points in three-dimensional space define a plane, it will be appreciated that the alignment of the three reference points 21′-23′ of the reference pattern image 20′ with the corresponding points 11-13 of the subject's jaw 10 will produce a three-dimensional alignment of the reference pattern with the subject's jaw. When the reference pattern is so aligned with the subject's jaw, the projected images 24′ of the guides 24 on the reference pattern will clearly indicate, at 14, the location, direction, and/or magnitude each working operation (e.g., drilling of a hole for receiving a dental implant) is to be performed in the subject's jaw.

A shutter system is used to faciliate the alignment of the reference pattern 20 with the subject's jaw 10. For this purpose, a first shutter 45 in interposed between face 41 of beam splitter 40 and the subject's jaw 10, and a second shutter 46 is interposed between face 42 and the 45° mirror 30 projecting the reference pattern 20. The two shutters 45, 46 may be alternately opened and closed at a sufficiently high speed so that the viewer, while viewing the subject's jaw via face 43 of beam splitter 40 during the alignment phase, will easily see, by persistence of vision, the image 20′ of reference pattern 20 superposed on the subject's jaw 10.

The two shutters 45, 46, may be mechanical shutters, such as rotating discs, driven by a control unit schematically shown at 47. The two shutters 45, 46, however, may also be liquid crystal shutters which are electronically controlled by the control unit 47. Where the object, with the superposed reference pattern, is viewed by means of an electronic camera an will be described below, the focus control of the camera may be used for the shutters. The two shutters may be alternately opened and closed during the alignment phase and may be left open during the working phase.

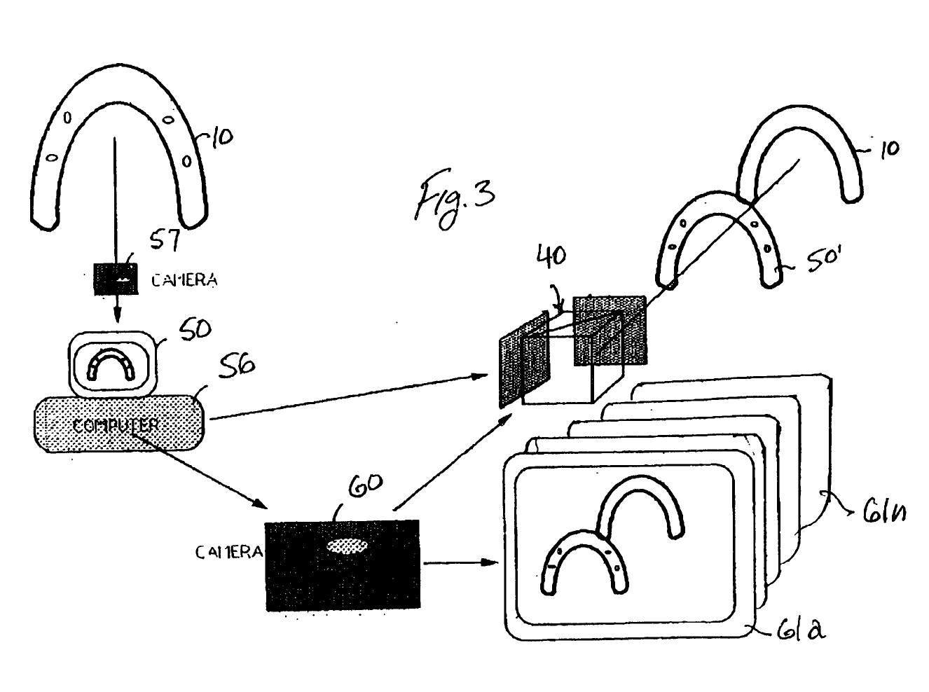

FIG. 2 illustrates another embodiment of the invention wherein the reference pattern, instead of being a physical pattern as shown at 20 in FIG. 1, is in the form of a computer generated image, generally designated 50. The reference pattern may be generated from the particular patient by any of the known techniques, such as CT (computerized topography), MRI (magnetic resonance imaging), or three-dimensional photographic techniques. The image may be generated in real time with the performance of the working operation, or it may be generated and stored in digital form and later used in the performance of the working operation.

The computer-generated image 50 also includes the three visually-discernible reference points 51, 52, 53. It may also include, or it may have applied to it as an overlayer, the visually-discernible guide 54 indicating the location, direction, and/or magnitude the working operation is to be performed on the object, e.g., the drilling of one or more holes in the subject's jaw for purposes of receiving a dental implant.

The reference pattern 50 in the apparatus of FIG. 2 is generated and manipulated by the computer 56 to align the three reference points 51-53 with the subject's jaw when superposed thereon, as described above with respect to FIG. 1. Since computer-generated images can be easily electronically manipulated, it is not necessary to include the 45° mirror 30 in the system of FIG. 1, which was provided to invert the image of the reference pattern so that it will be properly oriented with the subject's jaw when superposed thereon.

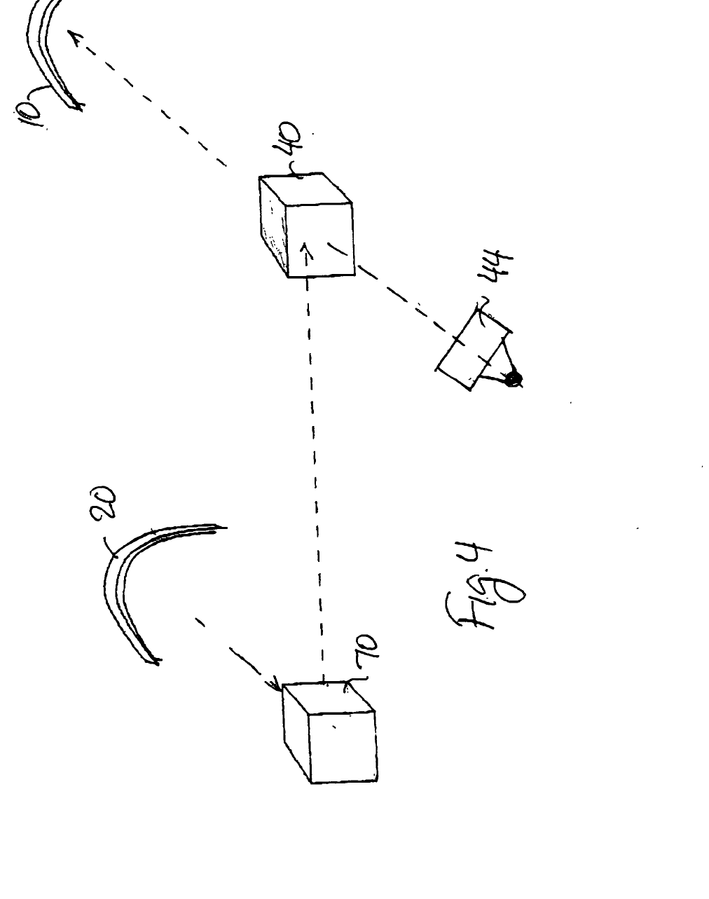

FIG. 3 illustrates a further variation wherein the apparatus includes an electronic 3-D camera 57 connected to the computer 56 for generating the reference pattern from the patient's jaw 10, and another electronic 3-D camera 60 which digitizes this information and displays it in one or more monitors 61a--61n. An advantage of this arrangement is that the viewed image can be simultaneously displayed in a plurality of monitors located at a plurality of stations for instructing a plurality of students with respect to the particular operation to be performed. For example, an instructor could perform the one or more critical working operations on the object 10, or a model of the object, to produce the reference pattern 50, while the operations of the instructor so produced are superposed on the object 10, or model thereof, assigned to each student located at a station equipped with a monitor 61a-61n, for guiding the students in the operations to be performed on their objects. The operations performed by an instructor could be recorded as course instructional material, or could be performed in a real time manner by the instructor with the performance by the students.

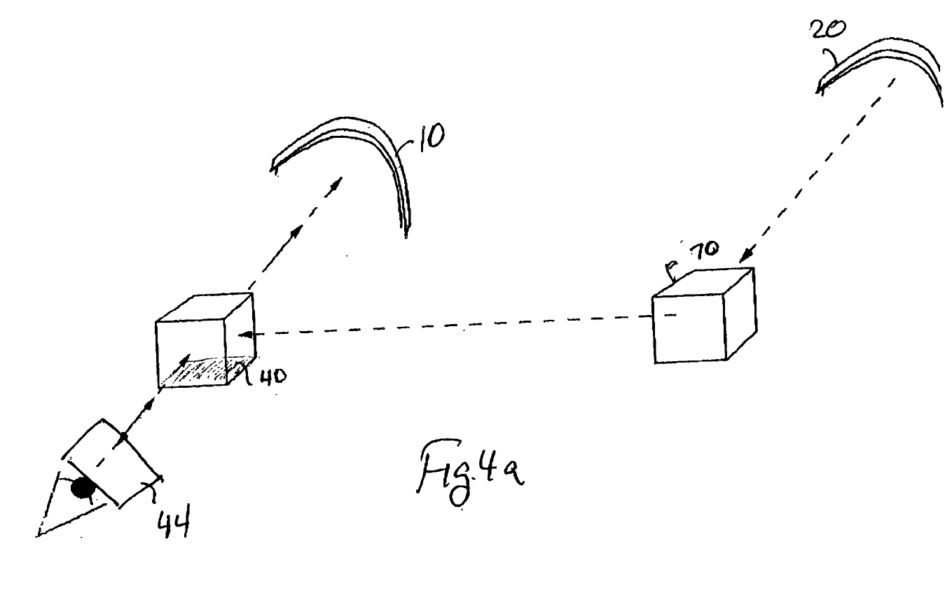

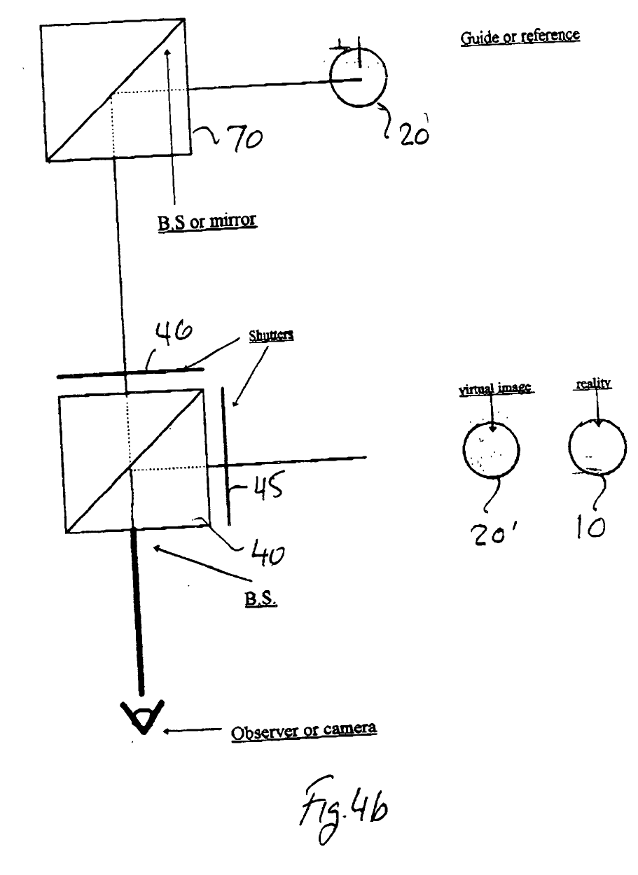

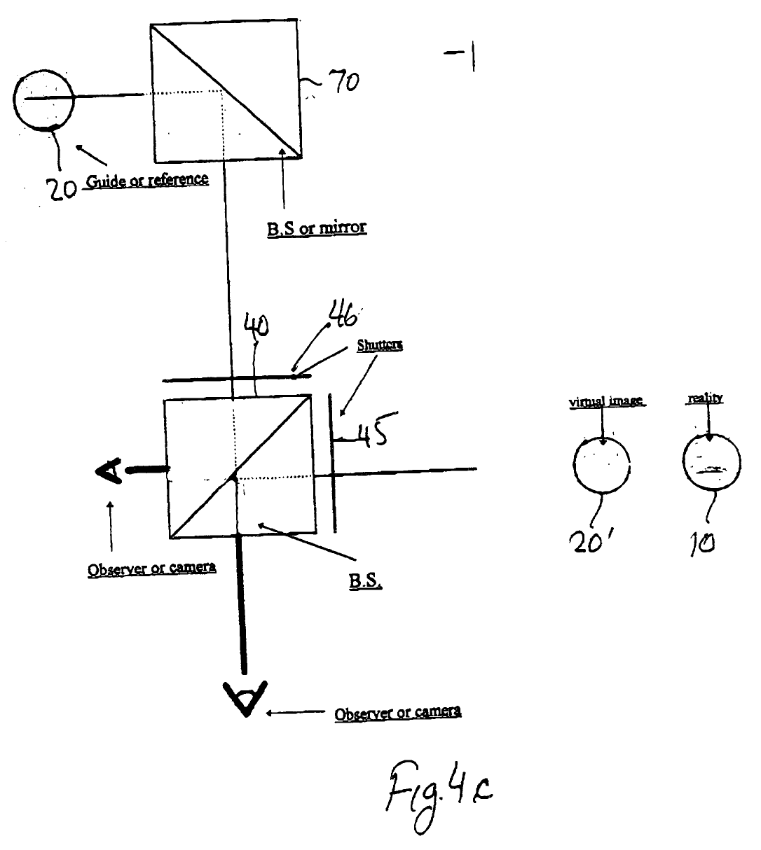

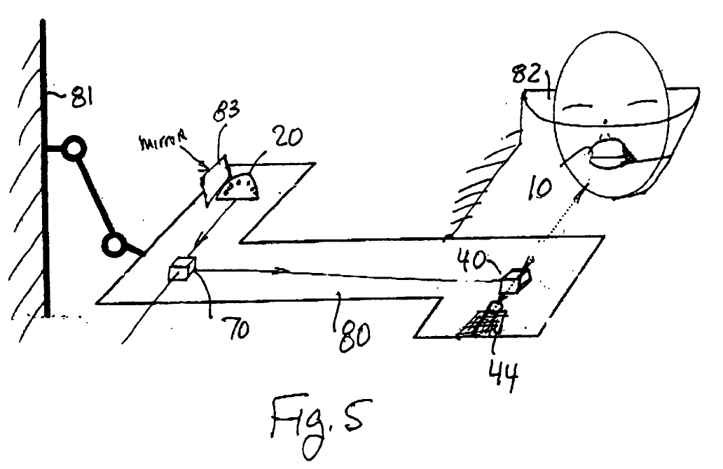

FIGS. 4 and 4a illustrate a variation wherein the 45° mirror 30 in FIG. 1 is replaced by a second beam splitter 70. An advantage of such an arrangement is that it provides great flexibility according to the particular situation. Thus, the user can view the object 10 from one side, via beam splitter 40 as shown in FIG. 4, or from the opposite side, by rotating the two beam splitters and the eyepiece 180°, as shown in FIG. 4a. FIG. 4b illustrates using another face of beam splitter 40 for viewing the object 10 with the superposed reference pattern 20′; and FIG. 4c illustrates using another face of beam splitter 20 for projecting the image of the reference pattern 20 on the object 10.

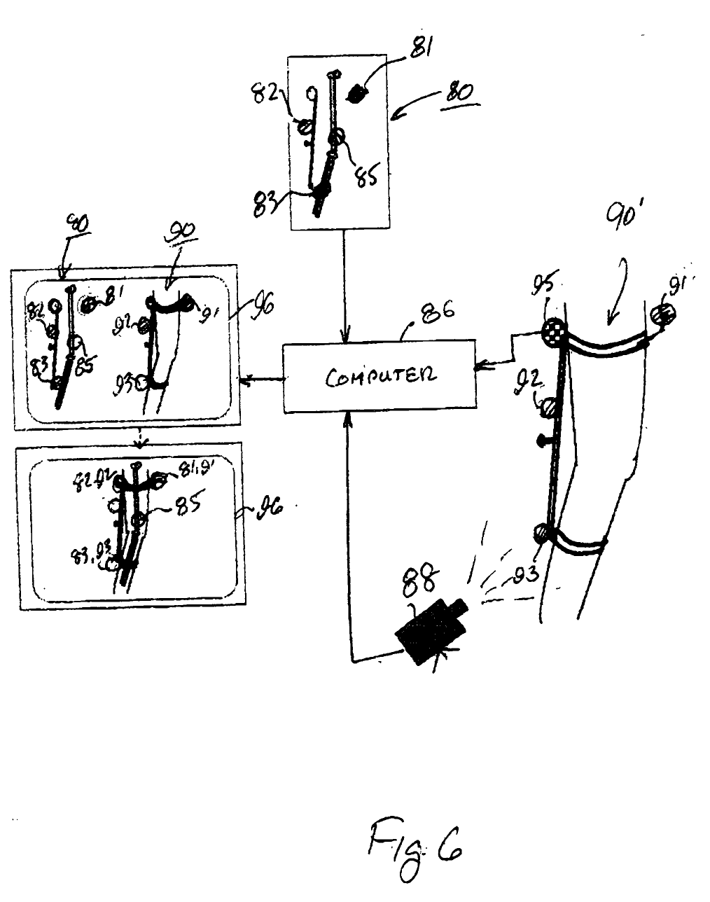

If the object to be worked on is a moving object, such as a body part of a patient (as distinguished from a non-moving object such as a industrial part to be manufactured or processed), it would be necessary to immobilize or secure the object with respect to the reference pattern and the viewing device during the alignment phase and also during the working phase. FIG. 5 schematically illustrates such an arrangement as applied to the two beam-splitter setup of FIGS. 4 and 4a. Thus, both beam splitters 40, 70 are carried by a common support 80 mounted in cantilever fashion from the side, as shown at 81 (or mounted from above, not shown). The subject's head is fixed with reference to the reference pattern 20 and the camera 44 by means of an anchoring member 82 which firmly engages the subject's head and is secured from the side (or from above) against all movement. Where the operation to be performed is a dental operation, such as drilling a hole in the subject's jaw for a dental implant, both jaws of the subject should be immobilized, which can be done by a chuck (not shown) inserted between the subject's jaws and firmly locking them in a fixed open position.

FIG. 5 also illustrate a further variation if a 2-D cammera 44 is used, rather than a 3-D camera. Thus, as shown in FIG. 5, a mirror 83 may be used to produce an image of the 2-D reference pattern 20 in a different plans from that of the camera in order to obtain the perception of depth.

As indicated earlier, whenever a camera is used for viewing the object with the superposed reference pattern, the manual focus control of the camera may be used for the shutters. Thus, focussing the image effectively “opens” the shutter, and defocussing the image effectively “closes” the shutter.

The apparatus illustrated in FIG. 5 is otherwise constructed and operates in substantially the same manner as described above.

FIG. 6 illustrates a still further arrangement, wherein the reference pattern is superposed, not on the physical object itself, but rather on an image of the physical object, and not by optical means, but rather by electronic means.

Thus, as shown in FIG. 6, a reference pattern, schematically indicated at 80, is generated in any known manner, e.g., by X-ray, CT or MIR. This reference pattern includes the three reference points 81, 82 and 83, respectively, and also the point of interest shown at 85. For example, the point of interest 85 may be a piece of shrapnel lodged in a patient's leg.

The reference pattern 80 is fed to a computer 86. Computer 86 also receives, via a camera 88, an image 90 of the actual physical object 90′, in this case the patient's leg. The physical object 90′ also carries the three corresponding reference points, shown at 91, 92, 93, respectively.

Thus, the computer 86 produces a display, shown at 96, of the reference image 80 and the image 90 of the actual physical object 90′. Either of these images may then be manipulated by the operator to superpose them and to align their corresponding reference points with each other, i.e., 81 with 91, 82 with 92 and 83 with 93. When the two images are thus superposed and aligned, it will be seen that the point of interest, namely element 85 in the reference pattern 80, will be superposed on the image 90 of the actual physical object 90′, thereby enabling the observer to see exactly the location of that element (e.g., shrapnel) in the subject's leg to faciliate its removal.

If the physical object 90′ being examined is not fixed in space, it is necessary to compensate, in the display of the reference pattern, for any movements in the physical object. This is done in the arrangement illustrated in FIG. 6 by providing an electrical gyro system, schematically indicated at 95, to detect any movements of the physical object and to feed such movements into the computer 86. The computer is thus able to make the appropriate changes in the spatial display of the reference pattern 80. Thus, the gyro detector 95 in FIG. 6 serves basically the same function as the mechanical coupling in FIG. 5, to compensate the spatial positioning of the reference pattern for any movements of the actual physical object being examined.

While the invention has been described with respect to several preferred embodiments, it will be appreciated that these are set forth merely for purposes of example. Thus, the invention could be used in other surgical operations, e.g., for other implantations, for precise incisioning, for precise suturing, etc. Also, the invention could be used in many non-surgical operations, e.g., manufacturing, repairing, and sculpturing operations. Many other variations, modifications and applications of the invention will be apparent.

What is claimed is:

1. A method of displaying a point of interest on an object, comprising:
preparing a reference pattern of said object, which reference pattern includes said point of interest in a visually-discernible manner on the object and further includes three visually-discernible reference points on said object;
superposing said reference pattern on said object;
effecting relative movement between said reference pattern and said object to optically align said three reference points on the reference pattern with three corresponding reference points on the subject;
and displaying said reference pattern superposed on said object such that said point of interest on the reference pattern is visually indicated on the object.

2. The method according to claim 1, wherein said object is a physical object, and said point of interest indicates the location, direction and/or magnitude a working operation is to be performed on the physical object.

3. The method according to claim 2, wherein said reference pattern is prepared in the form of a physical member having the configuration of said physical object, and relative moment is effected between said reference pattern and said object by manipulating said physical member.

4. The method according to claim 2, wherein said reference pattern is prepared in the form of a computer-generated image having the configuration of said physical object, and said reference pattern is manipulated by electronically manipulating said computer-generated image.

5. The method according to claim 2, wherein said reference pattern superposed on said object is displayed to a viewer via an eyepiece oriented to view said object with the reference pattern superposed thereon.

6. The method according to claim 2, wherein said reference pattern superposed on said object is displayed to a viewer via a monitor controlled by an electronic camera oriented to view said object with the reference pattern superposed thereon.

7. The method according to claim 5, wherein said reference pattern superposed on said object is displayed to the viewer via a beam splitter having a first face aligned with said object, a second face aligned with said optically-projected reference pattern, and a third face through which the object and the superposed reference pattern are viewed.

8. The method according to claim 7, wherein said apparatus further includes a first shutter between said first face of the beam splitter and said object, and a second shutter between said second face of the beam splitter and said optically-projected reference pattern.

9. The method according to claim 8, wherein said shutters are alternately actuated during the alignment of said reference pattern with said object.

10. The method according to claim 2, wherein said reference pattern superposed on said object is displayed at a single station to a single viewer for purposes of aiding the performance of said working operation on the object.

11. The method according to claim 2, wherein said reference pattern superposed on said object is displayed at a plurality of stations to a plurality of viewers for purposes of simultaneously instructing a plurality of persons in the performance of said working operation on the object.

12. The method according to claim 2, wherein said working operation a surgical operation.

13. The method according to claim 2, wherein said working operation is a dental operation.

14. The method according to claim 12, wherein said working operation is the drilling of a hole through a patient's bone for purposes of implanting a device, the visually-discernible guide of the reference pattern being in the form of a pin received within a hole indicating the location, orientation and depth of the hole to be drilled in the patient's bone.

15. The method according to claim 1, wherein said object is an image of the physical object.

16. The method according to claim 15, wherein said point of interest indicates the location of an element in the physical object, or the location, direction and/or magnitude a working operation is to be performed on the physical object.

17. The method according to claim 15, wherein said reference pattern is a pre-recorded image of the physical object containing the point of interest, and said object is a real-time image of the physical object not containing said point of interest.

18. The method according to claim 1, wherein said physical object is coupled to the display of said reference pattern such that after the superposition of the reference pattern image on said object, any movement of the physical object produces a corresponding movement of the reference pattern image.

19. The method according to claim 18, wherein said coupling is a mechanical coupling.

20. The method according to claim 18, wherein said coupling is an electrical coupling.

21. Apparatus for displaying a point of interest on an object, comprising:
a reference pattern of said object, which reference pattern includes said point of interest in a visually discernible manner on the object, and further includes three visually-discernible reference points on said object;
a superposing device for superposing said reference pattern on said object;
means for effecting relative movement between said reference pattern and said object to optically align said three reference points on the reference pattern with three corresponding reference points on the object;
and display means for displaying said reference pattern superposed on said object such that said point of interest on the reference pattern is visually indicated on said object.

22. The apparatus according to claim 21, wherein said object is a physical object, and said point of interest indicates the location, direction and/or magnitude a working operation is to be performed on the physical object.

23. The apparatus according to claim 22, wherein said reference pattern is in the form of a physical member having the configuration of said object, said reference pattern being movable with respect to said object by manipulating said physical member.

24. The apparatus according to claim 23, wherein said reference pattern is in the form of a computer-generated image having the configuration of said physical object, said reference pattern being manipulatable by electronically manipulating said computer-generated image.

25. The apparatus according to claim 22, wherein said display means includes an eyepiece oriented to view said object with the reference pattern superposed thereon.

26. The apparatus according to claim 22, wherein said display means includes an electronic camera oriented to view said object with the reference pattern superposed thereon, and a monitor controlled by said electronic camera to display said object with the reference pattern superposed thereon.

27. The apparatus according to claim 25, wherein said reference pattern superposed on said object is viewed via a beam splitter having a first face aligned with said object, a second face aligned with said optically-projected reference pattern, and a third face through which the object and the superposed reference pattern are viewed.

28. The apparatus according to claim 27, wherein said apparatus further includes a first shutter between said first face of the beam splitter and said object, and a second shutter between said second face of the beam splitter and said optically-projected reference pattern.

29. The apparatus according to claim 28, further including control means for alternately opening and closing said shutters.

30. The apparatus according to claim 27, wherein said apparatus further includes a 45° mirror between said reference pattern and said second face of the beam splitter.

31. The apparatus according to claim 27, wherein said apparatus further includes a second beam splitter between said reference pattern and said second face of the first-mentioned beam splitter.

32. The apparatus according to claim 22, wherein said display means displays said reference pattern superposed on said object at a single station for purposes of aiding the performance of said working operation on the object.

33. The apparatus according to claim 22, wherein said display means displays said reference pattern superposed on said object at a plurality of stations for purposes of simultaneously instructing a plurality of persons for performing said working operation on the object.

34. The apparatus according to claim 22, further including fixing means for fixing said object with respect to said reference pattern and said display means when said reference pattern is displayed superposed on said object.

35. The apparatus according to claim 34, wherein said fixing means fixes a body part of a subject with respect to said reference pattern and display means for performing a surgical operation on said subject.

36. The apparatus according to claim 34, wherein said fixing means fixes the head of a subject with respect to said reference pattern and display means for performing a dental working operation on said subject.

37. The apparatus according to claim 28, wherein said fixing means also fixes both jaws of the subject for drilling a hole in the subject's jaw to implant a dental device therein, the visually-discernible guide of the reference pattern being in the form of a pin received within a hole indicating the location, orientation and depth of the hole to be drilled in the patient's bone.

38. The apparatus according to claim 21, wherein said object is an image of the physical object.

39. The apparatus according to claim 38, wherein said point of interest indicates the location of an element in the physical object, or the location, direction and/or magnitude a working operation is to be performed on the physical object.

40. The apparatus according to claim 38, wherein said reference pattern is a pre-recorded image of the physical object containing the point of interest, and said object is a real-time image of the physical object not containing said point of interest.

41. The apparatus according to claim 21, wherein said physical object is coupled to the display of said reference pattern such that after the super-position of the reference pattern image on said object, any movement of the physical object produces a corresponding movement of the reference pattern image.

42. The apparatus according to claim 41, wherein said coupling is a mechanical coupling.

43. The apparatus according to claim 41, wherein said coupling is an electrical coupling.

44. The method of displaying the location, direction, and/or magnitude a working operation is to be performed on an object according to claim 1, substantially as described with reference to and as illustrated in the accompanying drawings.

45. The apparatus for displaying the location, direction, and/or magnitude a working operation is to be performed on an object according to claim 21, substantially as described with reference to and as illustrated in the accompanying drawings.
